(19) United States
(12) Patent Application Publication
(10) Pub. No.: US 2016/0058739 A1
(43) Pub. Date: Mar. 3, 2016

(54) USE OF PIDOTIMOD TO TREAT INFLAMMATORY BOWEL DISEASE

(71) Applicant: POLICHEM SA, Luxembourg (LU)

(72) Inventors: Federico Mailland, Lugano (CH); Francesco Scarci, Como (IT); Maurizio Caserini, Como (IT)

(21) Appl. No.: 14/781,796

(22) PCT Filed: Apr. 5, 2013
(86) PCT No.: PCT/EP2013/057208

§ 371 Date: Oct. 1, 2015

(51) Int. Cl. A61K 31/426; A61K 9/00; A61K 9/08; A61K 45/06

(52) U.S. Cl. CPC A61K 31/426; A61K 45/06; A61K 9/0053; A61K 9/08; A61K 9/0031

(57) ABSTRACT

The present invention is directed to the use of pidotimod, or a physiologically acceptable salt thereof, to treat inflammatory bowel disease. For the treatment of the present invention, pidotimod, or a physiologically acceptable salt thereof, may be administered either by oral route or rectally.

The present invention is directed to the use of pidotimod, or a physiologically acceptable salt thereof, to treat inflammatory bowel disease.

### BACKGROUND OF THE INVENTION

Inflammatory bowel disease (IBD) is a group of inflammatory conditions of the colon and small intestine. The major types of IBD are Crohn's disease (Crohn's) and ulcerative colitis (UC). Inflammatory bowel diseases are considered autoimmune diseases, in which the body's own immune system attacks elements of the digestive system. The main difference between Crohn's and UC is the location and nature of the inflammatory changes. Crohn's can affect any part of the gastrointestinal tract, from mouth to anus, although a majority of the cases start in the terminal ileum. UC, in contrast, is restricted to the colon and the rectum. Microscopically, ulcerative colitis is restricted to the mucosa (epithelial lining of the gut), while Crohn's affects the whole bowel wall (“transmural lesions”). Finally, Crohn's and UC present with extra-intestinal manifestations (such as liver problems, arthritis, skin manifestations and eye problems) in different proportions.

Although very different diseases, both may present with any of the following symptoms: abdominal pain, vomiting, diarrhea, rectal bleeding, severe internal cramps/muscle spasms in the region of the pelvis and weight loss. Anemia is the most prevalent extraintestinal complication of inflammatory bowel disease. Associated complaints or diseases include arthritis, pyoderma gangrenosum, and primary sclerosing cholangitis. Diagnosis is generally made by assessment of markers in stool followed by colonoscopy with biopsy of pathological lesions.

Inflammatory bowel disease, including Crohn's and ulcerative colitis, can be treated with a number of medications including 5-ASA drugs, such as Sulfasalazine and Mesalazine. Corticosteroids such as prednisone or budesonide can also be used due to their immunosuppressing and short term healing properties, but due to the risks outweighing the benefits, they are not used for long term treatment. Among corticosteroids, beclomethasone dipropionate may be effective for prolonged treatment in patients in the postacute phase (Prantera C., Therap Adv Gastroenterol. 2013;6(2):137-56). Immunosuppressive medications such as azathioprine, and biological agents such as infliximab and adalimumab are given lastly, only if patients cannot achieve remission with 5-ASA and corticosteroids, due to their rare but possible risk factors, including, but not limited to increased risk of cancers in teenagers and adults, tuberculosis and new or worsening heart failure (Danese S, et al. Aliment Pharmacol Ther. 2013 May;37(9):855-66.).

Pidotimod, whose chemical name is (4R)-3-(5-oxo-L-prolyl)-1,3-thiazolidine-4-carboxylic acid, was disclosed for the first time in 1T1231723. It is a synthetic dipeptide with capability to increase the immune response in animal models and in human beings. This compound has been shown to induce dendritic cell maturation and up-regulate the expression of HLA-DR and co-stimulatory molecules CD83 and CD86, which are integral to communication with adaptive immunity cells. Pidotimod has also been shown to stimulate dendritic cells to release pro-inflammatory molecules such as MCP-1 and TNF-α cytokines, and to inhibit thymocyte apoptosis caused by a variety of apoptosis inducing molecules.

Due to its capability to stimulate the immune system, pidotimod is believed to worsen those conditions characterized by an increased immune activity and its use is not recommended in such diseases.

It has now been surprisingly found that pidotimod, besides being active on illnesses characterized by immune defects, may be of benefit in patients with inflammatory bowel disease, by attenuating the symptoms including abdominal pain, vomiting, diarrhea, rectal bleeding, abdominal cramps and flatulence.

### DESCRIPTION OF THE INVENTION

The object of the present invention is represented by the use of pidotimod, or a physiologically acceptable salt thereof, for use in the treatment of inflammatory bowel diseases.

For the treatment of the present invention, pidotimod, or a physiologically acceptable salt thereof, may be administered either orally or rectally.

When administered orally, it may be in the form of solid or liquid formulations containing pidotimod or a physiologically acceptable salt thereof together with at least a pharmaceutically acceptable excipient and/or adjuvant; such formulations may be in the form of tablets, film-coated tablets, capsules, dragées, sachets, solutions or suspensions.

Such liquid formulations to be orally administered may have a w/w concentration in pidotimod from 0.5% to 20%, more preferably from 1% to 10%, most preferably from 2% to 8%.

Such solid formulations to be orally administered may have a w/w concentration in pidotimod from 50% to 90%, more preferably from 65% to 80%, most preferably from 70% to 75%.

According to an embodiment of the invention, when administered orally, the amount of pidotimod or of a physiologically acceptable salt thereof, may vary from 10 to 1000 mg per single dose, more preferably from 50 to 800 mg per single dose.

Such solid, semi-solid or liquid formulations are particularly suitable to treat inflammatory bowel disease in all its manifestations, including IBD-D, IBD-C and IBD-A.

When rectally administered, pidotimod, or a physiologically acceptable salt thereof, may be in the form of semi-solid or liquid formulations containing pidotimod or a physiologically acceptable salt thereof, together with at least a pharmaceutically acceptable excipient and/or adjuvant; such formulations may be in the form of enema, suppositories, solutions, emulsions or suspensions.

Such semi-solid or liquid formulations to be rectally administered may have a w/w concentration in pidotimod from 0.1% to 20%, more preferably from 1% to 15%, most preferably from 5% to 10%. They are particularly suitable to treat inflammatory bowel disease by direct application over the intestinal mucosa.

Pharmaceutical compositions may be prepared according to conventional techniques, may contain pharmaceutically acceptable excipients, adjuvants and/or carriers, and may also contain, in combination, one or more active principles with complementary or, in any case, useful activity.

The active agents which may be used in combination with pidotimod of the present invention include, but are not limited to, 5-ASA drugs, such as Sulfasalazine and Mesalazine, Corticosteroids such as prednisone, budesonide or beclomethasone dipropionate, immunosuppressive medications such as azathioprine, and biological agents such as infliximab and adalimumab.

Examples of the compositions prepared according to the present invention include: tablets, film-coated tablets, capsules, dragées, suspension or solutions suitable for oral administration; enema, suppositories, solutions, emulsions, suspensions for rectal application. The pharmaceutical compositions and the uses of the present invention will now be more fully described by the following examples. It should, however, be noted that such examples are given by way of illustration and not of limitation.

### EXAMPLE 1

A rectal solution having the following w/w % composition was prepared:

| | |
|---|---|
| 1. Pidotimod | 10.00% |
| 2. Tris(hydroxymethyl)methylamine | 5.00% |
| 3. Disodium EDTA | 0.10% |
| 4. Propylene Glycol | 5.00% |
| 5. Lactic acid | 0.15% |
| 6. Hydroxypropyl Chitosan | 1.00% |
| 7. Purified water | q.s. to 100.00% |

Preparation

Solubilize components 1, 2, 3, 4, 6 in water. Add component 7 and mix until clear solution is obtained.

### EXAMPLE 2

A rectal gel formulation having the following w/w % composition was prepared:

| 1. Purified water | q.s to 100.00% |
|---|---|
| 2. Pidotimod | 10.00% |
| 3. Tris(hydroxymethyl)methylamine | 5.00% |
| 4. Disodium Edta | 0.10% |
| 5. Glycerin | 5.00% |
| 6. 5-Ureidohydantoin | 0.30% |
| 7. Thickeners | 0.80% |
| 8. Hydroxypropyl Chitosan | 0.50% |
| 9. Preservatives | 0.33% |

Preparation

In the main vessel combine the components 1, 2, 3, 4, 5, 6, and 9. Mix until clear solution. Add thickeners homogenizing after each addition and until fully dispersed. Separately solubilize component 8 in part of water and add it in the main vessel while stirring. Mix until homogeneity.

### EXAMPLE 3

A granulate for oral administration having the following w/w % composition was prepared:

| | |
|---|---|
| 1. Pidotimod | 26.67% |
| 2. Mannitol | 3.33% |
| 3. Binder and wetting agent | 0.90% |
| 4. Sweetener | 0.60% |
| 5. Flavour | 16.67% |
| 6. Sodium carbonate anhydrous | 5.67% |
| 7. Silicon dioxide | 0.33% |
| 8. Colouring agents | 0.04% |
| 9. Saccharose | q.s. to 100% |

Preparation

In a vessel dissolve the component 3 in a suitable quantity of water. Mix until clear solution. In another vessel mix the components 1 and 2. Spray the obtained solution onto mixed components until a homogeneous granulate is obtained. After drying, components from 4 to 9 are added to the obtained granulate. All components are mixed until an homogeneous mixture is obtained.

### EXAMPLE 4

A solution for oral administration having the following w/w % composition was prepared:

| | |
|---|---|
| 1. Pidotimod | 5.10% |
| 2. Sodium chloride | 0.07% |
| 3. Sodium saccharin | 0.06% |
| 4. Chelating agents | 0.05% |
| 5. Tromethamine | 2.50% |
| 6. Preservatives | 0.15% |
| 7. Sorbitol solution | 31.89% |
| 8. Flavouring agents | 0.30% |
| 9. Antioxydants | 0.07% |
| 10. Colouring agents | 0.01% |
| 11. Purified water | 59.80% |

Preparation: in a vessel dissolve the components 1 to 10 in a suitable quantity of purified water. Mix until a clear solution is obtained. Add the remaining quantity of water, mix until a homogeneous solution is obtained and filter.

### EXAMPLE 5

A tablet for oral administration having the following w/w % composition was prepared:

| | |
|---|---|
| 1. Pidotimod | 72.70% |
| 2. Diluents | 17.65% |
| 3. Sodium Carboxymethyl cellulose crosslinked | 4.55% |
| 4. Binders | 4.00% |
| 5. Magnesium stearate | 1.10% |

In a vessel mix the components 1 and 2. In another vessel dissolve the component 4 in a suitable quantity of water. Mix until a clear solution is obtained. Spray the obtained solution onto mixed components I and 2 until a homogeneous granulate is obtained. After drying, components 3 and 5 are added to the obtained granulate and mixed until a homogeneous mixture is obtained. The mixture is then compressed by means of a tableting machine.

### EXAMPLE 6

Three patients with chronic diarrhoea caused by Crohn's disease without resection, aged 44 to 63 years (2 female) and three patients with chronic diarrhoea caused by ulcerative colitis, aged 50 to 65 years (1 female) were enrolled in an open-label pilot trial to receive twice a day the composition as per the Example 4 for 12 weeks. The frequency and weight of stools significantly decreased, the stools became more solid, and bowel transit time was prolonged during pidotimod treatment.

Conclusions: The result of this study showed that Pidotimod administered twice daily for 12 weeks has a beneficial role in inflammatory bowel disease (IBD) in controlling signs and symptoms such as chronic diarrhea.

1. Pidotimod or a physiologically acceptable salt thereof, for use in the treatment of inflammatory bowel disease.

2. Pidotimod or a physiologically acceptable salt thereof for use according to claim 1, characterized in that said inflammatory bowel disease is in form of Crohns' disease or ulcerative colitis.

3. Pidotimod or a physiologically acceptable salt thereof for use according to claim 1, characterized in that it is administered to a human.

4. Pidotimod or a physiologically acceptable salt thereof for use according to claim 1, characterized in that it is administered orally.

5. Pidotimod or a physiologically acceptable salt thereof for use according to claim 4, characterized in that it is administered by means of a solid or liquid formulation.

6. Pidotimod or a physiologically acceptable salt thereof for use according to claim 5, characterized in that said solid formulation is a tablet, a film-coated tablet, a capsule, a dragée or a sachet.

7. Pidotimod or a physiologically acceptable salt thereof for use according to claim 5, characterized in that said liquid formulation is a solution or a suspension.

8. Pidotimod or a physiologically acceptable salt thereof for use according to claim 5, characterized in that said solid formulation has a w/w concentration in pidotimod from 50% to 90%, more preferably from 65% to 80%, most preferably from 70% to 75%.

9. Pidotimod or a physiologically acceptable salt thereof for use according to claim 5, characterized in that said liquid formulation has a w/w concentration in pidotimod from 0.5% to 20%, more preferably from 1% to 10%, most preferably from 2% to 8%.

10. Pidotimod or a physiologically acceptable salt thereof for use according to claim 5, characterized in that said formulation has a content in pidotimod or a salt thereof, from 10 to 1000 mg per single dose, preferably from 50 to 800 mg per single dose.

11. Pidotimod or a physiologically acceptable salt thereof for use according to claim 1, characterized in that it is administered rectally.

12. Pidotimod or a physiologically acceptable salt thereof for use according to claim 11, characterized in that it is administered by means of a semi-solid or liquid formulation.

13. Pidotimod or a physiologically acceptable salt thereof for use according to claim 12, characterized in that semi-solid formulation is a suppository, a cream, a gel, an ointment or an emulsion.

14. Pidotimod or a physiologically acceptable salt thereof for use according to claim 12, characterized in that said liquid formulation is a solution or a suspension.

15. Pidotimod or a physiologically acceptable salt thereof for use according to claim 12, characterized in that said formulation has a w/w concentration in pidotimod or a salt thereof from 0.1% to 20%, preferably from 1% to 15%, more preferably from 5% to 10%.

16. Pidotimod or a physiologically acceptable salt thereof for use according to any of the preceding claims, characterized in that it is administered in combination or in temporal proximity with at least one additional active principle.

17. Pidotimod or a physiologically acceptable salt thereof for use according to claim 16, characterized in that said at least one additional active principle is selected from 5-ASA drugs, corticosteroids, immunosuppressive agents, and biologics.

18. Pidotimod or a physiologically acceptable salt thereof for use according to claim 17, characterized in that said at least one 5-ASA drug is selected from Sulfasalazine and Mesalazine.

19. Pidotimod or a physiologically acceptable salt thereof for use according to claim 17, characterized in that said at least one corticosteroid is selected from prednisone, budesonide or beclomethasone dipropionate.

20. Pidotimod or a physiologically acceptable salt thereof for use according to claim 17, characterized in that said at least immunosuppressive medication is azathioprine.

21. Pidotimod or a physiologically acceptable salt thereof for use according to claim 17, characterized in that said at least one biological agents is selected from infliximab and adalimumab.
